(19) United States
(12) Patent Application Publication
(10) Pub. No.: US 2004/0146585 A1
(43) Pub. Date: Jul. 29, 2004

(54) Use of thiol-based compositions in treating chemotherapeutic agent-induced thrombocytopenia

(75) Inventors: Edward A. Neuwelt, Portland, OR (US); Leslie L. Muldoon, Tigard, OR (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Oregon Health & Science University, Portland, OR

(21) Appl. No.: 10/698,955

(22) Filed: Oct. 31, 2003

Related U.S. Application Data
(60) Provisional application No. 60/423,349, filed on Oct. 31, 2002.

(51) Int. Cl. A61K 33/24; A61K 31/095

(52) U.S. Cl. 424/649; 514/706

(57) ABSTRACT

The present invention provides methods for preventing or reducing chemotherapeutic agent-induced thrombocytopenia. These methods comprise administering an effective amount of a thiol-containing compound or composition (e.g., sodium thiosulfate and N-acetylcysteine) to a subject in need thereof.

### CROSS-REFERENCE TO RELATED APPLICATION

This application claims the benefit of U.S. Provisional Patent Application No. 60/423,349 filed Oct. 31, 2002, which is incorporated herein by reference in its entirety.

### STATEMENT OF GOVERNMENT INTEREST

This invention was supported by National Institutes of Health Grants R01 NS 44697 and NS 33618 and a Veterans Administration Merit Review Grant. The government has certain rights in this invention.

### BACKGROUND OF THE INVENTION

1. Field of the Invention

The present invention is directed to a method for preventing or treating chemotherapeutic agent-induced thrombocytopenia. In particular, the invention is directed to administering a thiol-based compound or composition to prevent or reduce such a side effect.

2. Description of the Related Art

Thrombocytopenia remains a major problem in patients receiving chemotherapy. Clinical sequelae of severe reduction in platelets include the risk of hemorrhage. Platelet transfusions, the primary intervention for severe thrombocytopenia, have been associated with febrile or allergic transfusion reactions, bacterial and viral infections, and circulatory congestion. Additionally, platelet transfusions are expensive and supplies are often limited. Severe thrombocytopenia is a major dose limiting toxicity of certain chemotherpeutic agents (e.g., carboplatin) with standard interventions including dose reduction and delay in subsequent chemotherapeutic treatment cycles. Several agents have been used in an attempt to prevent chemotherapy-induced thrombocytopenia including recombinant human interleukin-11, recombinant human thrombopoietin, the full-length clone and the pegylated, truncated form, and interleukin-6. Although agents such as IL-11 and recombinant human thrombopoietin are effective in increasing platelet counts in patients undergoing chemotherapy, associated toxicities are of concern and these agents have not yet provided consistent results in the treatment or prophylaxis of thrombocytopenia. IL-11 has been associated with fluid retention and cardiac arrhythmias, whereas some patients treated with pegylated, truncated thrombopoietin have developed neutralizing antibodies.

### SUMMARY OF THE INVENTION

The present invention provides methods for preventing or ameliorating chemotherapeutic agent-induced thrombocytopenia. Such methods comprise administering to a patient in need thereof an effective amount of a thiol-based compound or composition.

The thiol-based compounds of the present invention may be administered intravenously, intra-arterially, intra-peritoneally, orally, intradermally, subcutaneously, or transdermally. In certain embodiments, the thiol-based compound is administered intravenously or intra-arterially.

In certain embodiments, the thiol-based compound is administered prior to the administration of the chemotherapeutic agent or at least one of the chemotherapeutic agents. In other embodiments, the thiol-based compound is administered concurrently with the administration of the chemotherapeutic agent or at least one of the chemotherapeutic agents. In certain embodiments, the thiol-based compound is administered following the administration of the chemotherapeutic agent or at least one of the chemotherapeutic agents. For instance, the thiol-based compound may be administered at least 30 minutes, 1 hour, 2 hours, 4 hours, 6 hours or 8 hours following the completion of the administration of the chemotherapeutic agent of agents.

The thiol-based compound or composition may be selected from a group consisting of sodium thiosulfate, N-acetyl cysteine, glutathione ethyl ester, D-methionine, S-adenosyl-methionine, cysteine, N,N′-diacetyl-cysterine, cystathione, glutathione, glutathione ethyl ester, glutathione diethyl ester, S-(1,2-dicarboxyethyl) glutathione triester, cysteamine, cysteine isopropylester, thiol amifostine and combinations thereof. In certain embodiments, the thiol-based compound or composition is sodium thiosulfate, N-acetyl cysteine, or combinations thereof.

The chemotherapeutic agent may be any compound that is administered to a mammalian subject to destroy, or otherwise adversely affect, cancer cells. They may be platinum derivatives, taxanes, steoid derivatives, anti-metabolites, plant alkaloids, antibiotics, arsenic derivatives, intercalating agents, alkylating agents, enzymes, biological response modifiers and combinations thereof. In certain embodiments, the chemotherapeutic agents are alkylating agents, such as platinum-containing alkylating agents. Exemplary platinum-containing alkylating agent may be cisplatin, carboplatin, oxyplatin, or combinations thereof. In certain embodiments, the chemotherapeutic agents comprise cyclophosphamide, carboplatin and etoposide phosphate.

A patient in need of prevention or amelioration of chemotherapeutic agent-induced thrombocytopenia may be a human, a non-human primate, or another mammal that will undergo (or is undergoing) chemotherapy and is at high risk (or is suffering from) a chemotherapeutic agent-induced thrombocytopenia. In certain embodiments, the patient may suffer from tumor in the head or neck (e.g., brain tumor or cancer). In other embodiments, the patient may suffer from tumor or cancer located other than head or neck. In certain embodiments, the patient receives a blood brain barrier disruption procedure. In other embodiments, the patient does not receive a blood brain barrier disruption procedure.

The dosage of using sodium thiosulfate, an exemplary thiol-based compound, in preventing or ameliorating thrombocytopenia may be at least 1, 2, 3, 4, 5, 6, 7, 8, 9, 10, 11, 12, 13, 14, 15, 16, 17, 18, 19, or 20 grams/m² in humans. In addition, multiple doses (e.g., 1, 2, 3, 4, 5, 6, 8, or 10) may be used.

### BRIEF DESCRIPTION OF THE DRAWINGS

FIG. 1 shows estimated probabilities of platelet nadir<20×10³/mm³ stratified by prior chemotherapy and KPS≧70.

FIG. 2 shows percent of patients and percent of carboplatin-based courses requiring platelet transfusion.

### DETAILED DESCRIPTION OF THE INVENTION

The present invention provides methods for preventing or reducing chemotherapeutic agent-induced thrombocytopenia. Such methods comprise administering to a patient in need thereof an effective amount of a thiol-based compound or composition, such as sodium thiosulfate and N-acetylcysteine.

As used therein, “thrombocytopenia” refers to a disorder in which the number of platelets is abnormally low (e.g., below 100 million per milliliter of blood). The platelet count in circulating blood is normally between 150 and 400 million per milliliter of blood. In certain patients, the number of platelets may be below 50 million per milliliter of blood. In patients with severe thrombocytopenia, the number of platelets may be below 20 million per milliliter of blood. Symptoms of thrombocytopenia include bruising, nosebleeds or oral bleeding, and petechial rash (pinpoint red spots). Mild thrombocytopenia can occur without symptoms.

“Chemotherapeutic agent” refers to a compound that is administered to a mammalian subject to destroy, or otherwise adversely affect, cancer cells. Chemotherapeutic agents include, but are not limited to, platinum derivatives (e.g., cisplatinum and carboplatinum), taxanes (e.g., paclitaxel), steoid derivatives, anti-metabolites (e.g., 5-fluorouracil, methotrexate and cytosine arabinoside), plant alkaloids (e.g., vindesine VP16, vincristine and vinblastine), antibiotics (e.g., adriamycin, mitomycin C, bleomycin, mithramycin, daunorubicin, mitoxantrone, and doxorubicin), etoposide, arsenic derivatives, intercalating agents, alkylating agents (e.g., melphalan, cyclophosphamide, chlorambucil, busulphan, thiotepa, isofamide, mustine, and the nitrosoureas), enzymes (e.g., asparaginase), biological response modifiers (e.g., immunoadjuvants and immunorestoratives), hydroxyurea, procarbazine, and combination thereof. In certain embodiments, chemotherapeutic agents are alkylating agents. In certain embodiments, alkylating agents are platinum-containing alkylating agents (e.g., cisplatin, carboplatin, and oxyplatin).

“Chemotherapeutic agent-induced thrombocytopenia” (interchangeably used with “chemotherapy-induced thrombocytopenia”) refers to thrombocytopenia caused or induced by the administration of a chemotherapeutic agent or a combination of chemotherapeutic agents.

“Preventing a chemotherapeutic agent-induced thrombocytopenia” refers to preventing or diminishing the occurrence of chemotherapeutic agent-induced thrombocytopenia. A subject in need of prevention of chemotherapeutic agent-induced thrombocytopenia refers to a human, non-human primate or other mammal that will undergo, or is undergoing, chemotherapy and is at high risk for chemotherapy-induced thrombocytopenia.

A subject at high risk for chemotherapy-induced thrombocytopenia is one that has at least one of the risk factors for chemotherapy-induced thrombocytopenia. Such risk factors include previous bone-marrow depleting chemotherapy, performance status greater than 1, platelet count less than 150,000/μl at day 1 before the initiation of chemotherapy, lymphocyte count less or equate to 700/μl at day 1 before the initiation of chemotherapy, polymorphonuclear leukocyte count less than 1,500/μl at day 1 before the initiation of chemotherapy, and undergoing high risk chemotherapy (Blay et al., Blood 92: 405-10, 1998). High risk chemotherapy refers to regimens containing greater than 90 mg/m² doxorubicin, greater than 90 mg/m² epirubicin, greater than 100 mg/m² cisplatin, greater than 9 g/m² ifosfamide, greater than 1 g/m² cyclophosphamide, greater than 500 mg/m² etoposide, or greater than 1 g/m² cytarabine per course (Blay et al., Blood 92: 405-10, 1998; Blay et al., Proc. Am. Soc. Clin. Oncol. 16: 56a, 1997; Blay et al., J. Clin. Oncol. 14: 636, 1996). In certain embodiments, a subject in need of prevention of chemotherapeutic agent-induced thrombocytopenia has one, two, three, four, five or more risk factors as described above.

“Ameliorating chemotherapeutic agent-induced thrombocytopenia” refers to reducing the severity of chemotherapeutic agent-induced thrombocytopenia. A subject in need of ameliorating a chemotherapeutic agent-induced thrombocytopenia refers to a human, non-human primate or other animal that is undergoing chemotherapy and suffers from a chemotherapeutic agent-induced thrombocytopenia.

“Thiol-based compound” refers to a compound containing a thio, thiol, aminothiol or thioester moiety. Thiol-based compounds include, but are not restricted to, sodium thiosulfate, N-acetyl cysteine, glutathione ethyl ester, D-methionine, S-adenosyl-methionine, cysteine, N,N′-diacetyl-cysterine, cystathione, glutathione, glutathione ethyl ester, glutathione diethyl ester, S-(1,2-dicarboxyethyl) glutathione triester, cysteamine, cysteine isopropylester, and thiol amifostine (Ethyol or WR 2721). Thiol-based compound of the present invention may be used individually or in combination with one or more other thiol-based compounds, and/or other pharmaceutical agents and excipients.

“Thiol-based composition” refers to a composition comprising at least one thiol-based compound. Such compositions may also include, in addition to one or more thiol-based compounds, pharmaceutically acceptable carriers that facilitate administration of thiol-based compound(s) to a mammalian subject.

The term “effective amount” refers to an amount of thiol-based compound or composition that is sufficient to prevent or reduce chemotherapeutic agent-induced thrombocytopenia.

The present application provides thiol-based compositions and methods for using such compositions in preventing or ameliorating chemotherapy-induced thrombocytopenia. Techniques for the formulation and administration of the compounds of the present application may be found in “Remington's Pharmaceutical Sciences” Mack Publishing Co., Easton, Pa., latest edition.

The thiol-based compounds of the present invention are formulated to be compatible with their intended route of administration. Examples of route of administration include intravenous (i.v.), intra-arterial (i.a.), intra-peritoneal (i.p.), oral (p.o.), intradermal, subcutaneous, and transdermal administration. Solutions or suspensions used for intravenous, intra-arterial, intradermal, or subcutaneous application can include one or more of the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. In addition, pH may be adjusted with acids or bases, such as hydrochloric acid or sodium hydroxide. The free radical scavengers are preferably administered in their un-oxidized form. The parenteral preparation can be enclosed in ampoules, disposable syringes or multiple dose vials made of glass or plastic.

Thiol-based compounds suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against contamination from microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In certain embodiments, thiol-based compounds (e.g., sodium thiosulfate) are administered i.v. This route of administration is useful in preventing or ameliorating thrombocytopenia induced by chemotherapy for treating head or neck tumor and brain cancer. Intravenous administration of thiol-based compounds (e.g., sodium thiosulfate and N-acetylcysteine) results in minimum amount of thiol-based compounds in brain due to the blood brain barrier, which in turn prevents or reduces neurotoxicity of these thiol-based compounds and adverse effects of these compounds on chemotherapy efficiency.

It is advantageous to formulate compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. While compounds that exhibit toxic side effects may be used, care should be taken to design a delivery system that targets such compounds to the site of affected tissue to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Various animal models and clinical assays for evaluating effectiveness of a particular thiol-based compound in preventing or reducing thrombocytopenia known in the art may be used in the present invention. They include, but are not limited to, those described in Blay et al., Blood 92: 405-10, 1998; Case et al., Stem Cells 18: 360-5, 2000; Issacs et al., J. Clin. Oncol. 15: 3368-77, 1997; Harker et al., Blood 89: 155-65, 1997. Additional assays are described in the examples below.

The dosage of using sodium thiosulfate in preventing or ameliorating thrombocytopenia, when administered intravenously, may be at least 1, 2, 3, 4, 5, 6, 7, 8, 9, 10, 11, 12, 13, 14, 15, 16, 17, 18, 19, or 20 grams/m² in humans, or a dosage in another subject comparable to that in humans. A dosage (“dosage X”) of a thiol-based compound in a subject other than a human is comparable to a dosage (“dosage Y”) of the thiol-based compound in humans if the serum concentration of the scavenger in the subject post administration of the compound at dosage X is equal to the serum concentration of the compound in humans post administration of the compound at dosage Y. In certain embodiments, sodium thiosulfate may be administered multiple times (e.g., 20 grams/m² 4 hours after the administration of a chemotherapeutic agent such as carboplatin followed by 16 grams/m2 8 hours after the administration of the chemotherapeutic agent). In certain embodiments, sodium thiosulfate may be administered in combination with another thiol-based compound such as N-acetylcysteine.

Thiol-based compounds may be administered to a subject in need thereof prior to, concurrent with, or following the administration of chemotherapeutic agents. For instance, thiol-based compounds may be administered to a subject at least 8 hours, 7 hours, 6 hours, 5 hours, 4 hours, 3 hours, 2 hours, 1.5 hours, 1 hour, or 30 minutes before the starting time of the administration of chemotherapeutic agent(s). In certain embodiments, they may be administered concurrent with the administration of chemotherapeutic agent(s). In other words, in these embodiments, thiol-based compounds are administrated at the same time when the administration of chemotherapeutic agent(s) starts. In other embodiments, thiol-based compounds may be administered following the starting time of administration of chemotherapeutic agent(s) (e.g., at least 30 minutes, 1 hour, 2 hours, 3 hours, 4 hours, 5 hours, 6 hours, 7 hours or 8 hours after the starting time of administration of chemotherapeutic agents). Alternatively, thiol-based compounds may be administered at least 30 minutes, 1 hour, 2 hours, 3 hours, 4 hours, 5 hours, 6 hours, 7 hours or 8 hours after the completion of administration of chemotherapeutic agents. Generally, these thiol-based compounds are administered for a sufficient period of time so that thrombocytopenia is prevented or reduced. Such sufficient period of time may be identical to, or different from, the period during which chemotherapeutic agent(s) are administered. In certain embodiments, multiple doses of thiol-based compounds are administered for each administration of a chemotherapeutic agent or a combination of multiple chemotherapeutic agents.

In certain embodiments, an appropriate dosage of a thiol-based compound (e.g., sodium thiosulfate) is combined with a specific timing and/or a particular route to achieve the optimum effect in preventing or reducing thrombocytopenia. For instance, sodium thiosulfate may be administered to a human at 15-20 grams/m² (e.g., 16 or 20 grams/m²), via i.v., at least 1 hour, 2 hours, 3 hours, 4 hours, 5 hours, 6 hours, 7 hours or 8 hours after the beginning, or the completion, of the administration of a chemotherapeutic agent or a combination of chemotherapeutic agents.

### EXAMPLES

### Example 1

Blood/bone marrow toxicity data from patients with malignant brain tumors treated with carboplatin-based regimens with or without delayed high-dose STS were graded in accordance with National Cancer Institute Common Toxicity Criteria (NCI CTC) (version 2.0).

Treatment Regimen. Patients with a variety of malignant brain tumor histologies, including metastases to the brain, were eligible for treatment. Patients who had previously undergone central nervous system or systemic radiotherapy or chemotherapy were eligible, however 2 weeks must have elapsed since prior radiotherapy and 4 weeks since prior chemotherapy. Patients were required to have normal hematologic, renal and hepatic function. Osmotic opening of the blood-brain barrier was performed with the patient under general anesthesia, thus patients in the BBBD treatment groups were required to have adequate pulmonary and cardiac function to tolerate anesthesia.

The BBBD procedure was done on two consecutive days, approximately every 4 weeks for up to 12 months. Mannitol (25%) was infused (5-10 cc/sec) into the appropriate internal carotid or vertebral artery, depending on tumor location, for 30 seconds. The combination chemotherapy regimen consisted of cyclophosphamide (330 Mg/m²/day×2 days; total dose, 660 mg/m²) (i.v.) beginning approximately 20 minutes before the mannitol infusion, carboplatin (200 mg/m²/day×2 days; total dose, 400 Mg/m²) (i.a.) administered over 10 minutes, within 5 minutes after the mannitol, and etoposide (i.v.) or etoposide phosphate (200 mg/m²/day×2 days; total dose, 400 mg/m²) (i.a. or i.v.). Patients received granulocyte-colony stimulating factor (G-CSF) (5 mcg/kg) for 10 days after chemotherapy or until the absolute neutrophil count was greater than 1000/μL.

STS was available as a 25% (250 mg/ml) solution. Dose was determined (16 or 20 grams/m²) and mixed with an equivalent amount of sterile water (1 ml: 1 ml) for infusion. Because high-dose STS (16 or 20 grams/m²) causes transient hypematremia, hypertension, and controllable grade II nausea and vomiting (National Cancer Institute CTC, version 2.0), patients were premedicated with antiemetics before STS. The most commonly used regimen was benadryl (12.5 mg), dexamethasone (6 mg), and if needed, ativan (0.5-1.0 mg), i.v. 30-45 minutes before STS.

STS was administered i.v. over about 15 minutes. The number of STS doses the patient received depended on the patient baseline hearing status, and whether they experienced an ototoxic shift during treatment. Patients treated with BBBD/STS received STS in one dose (20 grams/m²) 4 hours after carboplatin (n=10), or two doses (20 grams/m² at 4 hrs, and 16 grams/m² at 8 hours) after carboplatin (n=19).

Because malignant brain tumor patients are at risk for intracerebral hemorrhage, patients underwent platelet transfusion when platelet count was <20×10³/mm³. Following a platelet transfusion, on subsequent chemotherapy courses the carboplatin dose was reduced 25%.

Data Analysis. Blood/bone marrow toxicity data were graded in accordance with NCI CTC and analyzed in the following three treatment groups: 1) patients treated with carboplatin, cyclophosphamide and etoposide with BBBD and without STS, and 2) patients treated with carboplatin, cyclophosphamide and etoposide phosphate with BBBD and with STS. For both groups, blood/bone marrow toxicity data from all carboplatin-based courses of patients in treatment were included in the analysis.

Several analytic approaches were used for these data. First, platelet nadirs were plotted for each subject and as means for each of the three study groups. For group comparisons, a mixed, repeated measures analysis of variance (ANOVA) model was fit using the parameter platelet nadir for each course as the measure. Gender, KIDS (≧70 or not), prior chemotherapy, prior radiation, and age at first course were included as covariates. Several other events were examined including grade 3 or 4 platelet toxicity as defined by NCI CTC, platelet transfusion, and the need for hematologically driven chemotherapy dose reduction. Platelet nadir<20×10³/mm³, which has important clinical implications for patients in this treatment program, was also examined. For present/absent outcomes as these, a mixed model repeated measures analysis (using the present/absent outcomes as the best response for each course) was performed. The mixed model ANOVA's using presence/absence of the outcome was adjusted for the covariates listed above.

Analyses were performed using Version 8.01 of SAS® for Windows. The binary mixed model analyses were performed using the MACRO GLIMMIX written by the SAS Institute. For the covariates, backwards elimination was performed to eliminate variables that did not make a significant contribution to the prediction. P-value>0.20 was used as the criteria to remove a variable.

Results. Baseline patient characteristics are shown in Table 1.

TABLE 1
Baseline patient characteristics

| | BBBD/No STS | BBBD/STS |
|---|---|---|
| Characteristics | n = 24 | n = 29 |
| Age, years | | |
| Median | 30 | 44 |
| Minimum | 2 | 1 |
| Maximum | 69 | 63 |
| KPS | | |
| Median | 80 | 80 |
| Minimum | 50 | 40 |
| Maximum | 100 | 100 |
| Gender, % of group | | |
| Female | 42 | 28 |
| Male | 58 | 72 |
| Prior Chemotherapy, | | |
| % of group | | |
| Yes | 67 | 34 |
| No | 33 | 66 |
| Prior Radiotherapy, | | |
| % of group | | |
| Yes | 25 | 21 |
| No | 75 | 79 |
| Tumor Classification, | | |
| % of group | | |
| 1 | 8 | 24 |
| 2 | 17 | 3 |
| 3 | 4 | 34 |
| 4 | 13 | 14 |
| 5 | 58 | 24 |

KPS: Karmofsky Performance Status
Tumor classification:
1 = glioma, grade 3, 4
2 = CNS metastases
3 = oligodendroglioma (includes anaplastic oligodendroglioma, oligo-astrocytoma)
4 = recurrent primary CNS lymphoma
5 = miscellaneous (includes pnet, pineoblastoma, pineocytoma, brain stem glioma, germ cell, low grade glioma)

Twenty four patients underwent 125 courses of carboplatin, cyclophosphamide, and etoposide in conjunction with BBBD, without STS. Twenty nine patients underwent 129 courses of carboplatin, cyclophosphamide and etoposide phosphate with BBBD and with STS. Of the 29 patients treated with BBBD and with STS, 19 (66%) received two doses of STS.

Percent of patients with platelet nadir<20×10³/mm³, percent undergoing platelet transfusion and percent undergoing dose reduction of carboplatin are shown in Table 2.

TABLE 2
Percent of Patients and Percent of Carboplatin Courses
Requiring Platelet Transfusion and Dose Reduction.

| | Platelet Nadir | Platelet | |
|---|---|---|---|
| Treatment Group | <20 × 10³/mm³ | Transfusions | Dose Reductions |
| BBBD/no STS | | | |
| n = 24 | 25% pts | 33% pts | 33% pts |
| 125 courses | 7% courses | 9% courses | |
| BBBD/STS | | | |
| n = 29 | 4% pts | 7% pts | 0% pts |
| 129 courses | 1% courses | 2% courses | |

i.a = intra-arterial
pts = patients

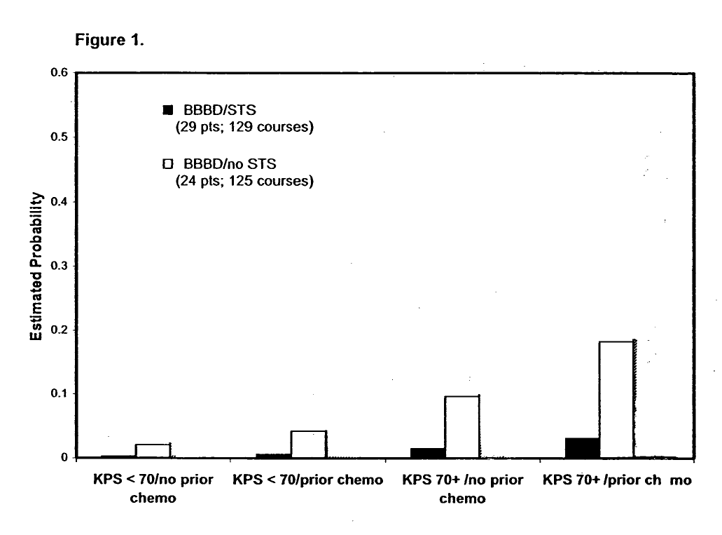

A mixed-model analysis of variance using the binary response of whether or not the platelet nadir was <20×10³/mm³ evaluated treatment group and course number controlling for KPS≧70 and prior chemotherapy. There was a significant difference among the two treatment groups (p=0.0002). There was no significant difference among the courses (p=0.33). KPS≧70 was statistically significant (p=0.040) while prior chemotherapy was not significant (p=0.087) but was left in the model as an adjusted variable. Both of these covariates had positive association with the probability of having platelet nadir<20×10³/mm³. In the case of KPS, patients with KPS less than 70 typically had fewer courses of chemotherapy (median of 2 compared with median of 4 for patients with KPS>70) so were actually less likely to have platelet nadirs<20×10³/mm³. The different between the two treatment groups was not significant (adjusted p=0.20). Estimated probabilities of platelet nadirs<20×10³/mm³ stratified by prior chemotherapy and KPS>70 are summarized in FIG. 1.

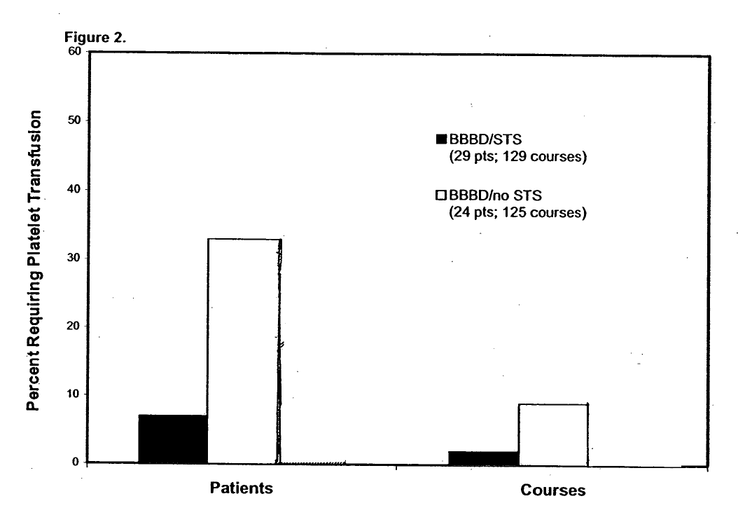

For the analysis of platelet transfusion (present or absent), none of the potential confounding variables was significant. There was a significant difference between the treatment groups (p=0.035) but no significant difference among the courses (p=0.11). The Bonferroni-adjusted comparison between the treatment groups is shown in FIG. 2. The difference between the treatment groups was not significant (p>0.10). The estimated probabilities of platelet transfusions were 9.0% for patients treated with BBBD and STS, and 22.4% for patients treated with BBBD without STS.

For grade 3 or 4 platelet toxicity, there was no significant difference among the treatment groups (p=0.14). There was a significant difference among the courses (p=0.0028). Both of these tests are adjusted for age at first carboplatin treatment (p=0.036). For grade 3 or 4 white blood cell toxicity, there was no significant difference among treatment groups or among courses.

The analysis of whether or not the patient required dose reduction of carboplatin is based on comparing rates of dose reduction using the Pearson chi-square test. The difference between the treatment groups was significant. Because no patients in BBBD with STS required dose reduction of carboplatin, model-based adjustment for covariates was not implemented for dose reductions.

### Example 2

A clinical study will include administering carboplatin (i.a.), cyclophosphamide (i.v.) and etoposide phosphate (i.v.), in patients with high grade glioma that do not receive BBBD. Patients will be randomized to receive delayed (4 and 8 hours after chemotherapy), high-dose (e.g., 20 grams/m²) STS (i.v.) or no STS. Primary endpoints will be percent of patients with platelet nadir<20×10³/mm³, and percent of patients requiring platelet transfusion. Tumor response and duration of response will be monitored. It will be determined whether STS prevents or treats thrombocytopenia.

### Example 3

Whether an optimized bone marrow chemoprotection regimen impaired the efficacy of enhanced chemotherapy against rat brain tumors was evaluated. Nude rats with intracerebral human lung carcinoma xenografts were treated with carboplatin, melphalan, and etoposide phosphate delivered intra-arterially with osmotic blood-brain barrier disruption (n=8 per group). Pretreatment with N-acetylcysteine combined with delayed administration of sodium thiosulfate protected against toxicity toward white cells and platelets (P=0.0016). The chemotherapy protocol significantly reduced intracerebral tumor volume (P<0.0001) and was unaffected by optimized chemoprotection. Negative interactions of thiols with anti-tumor efficacy were avoided by temporal and spatial separation of chemoprotectants and chemotherapy.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method for preventing or ameliorating chemotherapeutic agent-induced thrombocytopenia, comprising administering to a patient in need thereof an effective amount of a thiol-based compound or composition prior to, concurrently with, or following the administration of a chemotherapeutic agent or chemotherapeutic agents.

2. The method according to claim 1 wherein the patient in need thereof does not receive a blood brain barrier disruption procedure.

3. The method according to claim 1 wherein the thiol-based compound is administered intravenously.

4. The method according to claim 1 wherein the thiol-based compound is administered intra-arterially.

5. The method according to claim 1 wherein the thiol-based compound is administered prior to the administration of the chemotherapeutic agent or at least one of the chemotherapeutic agents.

6. The method according to claim 1 wherein the thiol-based compound is administered concurrently with the administration of the chemotherapeutic agent or at least one of the chemotherapeutic agents.

7. The method according to claim 1 wherein the thiol-based compound is administered following the administration of the chemotherapeutic agent or at least one of the chemotherapeutic agents.

8. The method according to claim 7 wherein the thiol-based compound is administered at least 30 minutes following the completion of the administration of the chemotherapeutic agent or at least one of the chemotherapeutic agents.

9. The method according to claim 7 wherein the thiol-based compound is administered at least 1 hour following the completion of the administration of the chemotherapeutic agent or at least one of the chemotherapeutic agents.

10. The method according to claim 7 wherein the thiol-based compound is administered at least 2 hours following the completion of the administration of the chemotherapeutic agent or at least one of the chemotherapeutic agents.

11. The method according to claim 7 wherein the thiol-based compound is administered at least 4 hours following the completion of the administration of the chemotherapeutic agent or at least one of the chemotherapeutic agents.

12. The method according to claim 7 wherein the thiol-based compound is administered at least 6 hours following the completion of the administration of the chemotherapeutic agent or at least one of the chemotherapeutic agents.

13. The method according to claim 7 wherein the thiol-based compound is administered at least 8 hours following the completion of the administration of the chemotherapeutic agent or at least one of the chemotherapeutic agents.

14. The method according to claim 1 wherein the thiol-based compound is selected from the group consisting of sodium thiosulfate, N-acetylcysteine, glutathione ethyl ester, glutathione, D-methionine, cysteramine, cystamine, aminopropylmethylisothiourea, and Ethyol, and combinations thereof.

15. The method according to claim 1 wherein the thiol-based compound is sodium thiosulfate.

16. The method according to claim 1 wherein the thiol-based compound is N-acetylcysteine.

17. The method according to claim 1 wherein the thiol-based composition comprises sodium thiosulfate and N-acetylcysteine.

18. The method according to claim 1 wherein the chemotherapeutic agent is an alkylating agent.

19. The method according to claim 17 wherein the alkylating agent is a platinum-containing alkylating agent.

20. The method according to claim 18 wherein the platinum-containing alkylating agent is selected from the group consisting of cisplatin, carboplatin, and oxyplatin.

21. The method according to claim 1 wherein the chemotherapeutic agents comprise cyclophosphamide, carboplatin and etoposide phosphate.

22. The method according to claim 1 wherein the patient in need thereof has a tumor in the head or neck.

23. The method according to claim 1 wherein the patient in need thereof is a human.

24. The method according to claim 23 wherein the thiol-based compound is sodium thiosulfate.

25. The method according to claim 24 wherein the chemotherapeutic agents comprise cyclophosphamide, carboplatin and etoposide phosphate.

26. The method according to claim 24 wherein sodium thiosulfate is administered at a dosage of 15-20 grams/m².

27. The method according to claim 26 wherein sodium thiosulfate is administered intravenously.

28. The method according to claim 27 wherein sodium thiosulfate is administered at least 4 hours following the administration of the chemotherapeutic agent or at least one of the chemotherapeutic agents.

29. The method according to claim 1 wherein the patient in need thereof has cancer other than brain tumor.
